(19) United States
(12) Patent Application Publication
(10) Pub. No.: US 2023/0167399 A1
(43) Pub. Date: Jun. 1, 2023

(54) METHOD FOR THE CONTINUOUS GENERATION AND HARVESTING OF BIOTHERMAL ENERGY

(71) Applicant: VRM International Pty Ltd, Garbutt (AU)

(72) Inventor: Kenneth Michael BELLAMY, Garbutt (AU)

(21) Appl. No.: 17/536,665

(22) Filed: Nov. 29, 2021

(51) Int. Cl. C12M 1/00; C12M 1/02; F28D 20/00

(52) U.S. Cl. CPC C12M 43/08; C12M 41/24; F28D 20/003

(57) ABSTRACT

A method for generating and capturing biothermal energy comprising: forming a heap comprising an amended organic material; subjecting the amended organic material to a continuous fermentation process to produce a convection current, and to stimulate capture of non-visible radiation, and using a heat exchanger in contact with the heap, capture and/or store biothermal energy generated by the continuous fermentation process within the heap.

### TECHNICAL FIELD

The present invention relates to a method for the continuous generation and harvesting of biothermal energy. In particular, the method relates to the continuous generation and harvesting of biothermal energy from an organic material subjected to a continuous fermentation process which relies at least in part on the capture and processing of non-visible radiation.

### BACKGROUND

Many methods and apparatus are known in the art for the purpose of decomposing, in an economical and environmentally acceptable manner, organic materials, by anaerobic fermentation and/or aerobic decomposition and of simultaneously producing an organic, or partially organic, output product.

However, while it is known that during high-temperature phases (~60° C.) of municipal waste composting, on average 1136 kJ kg⁻¹ of heat is released, limited studies have been reported on compost heat reuse (G. Irvine, E. R. Lamont, B. Antizar-Ladislao, “Energy from Waste: Reuse of Compost Heat as a Source of Renewable Energy”, International Journal of Chemical Engineering, vol. 2010, Article ID 627930, 10 pages, 2010. https://doi.org/10.1155/2010/627930). One such study was undertaken by the University of Edinburgh (Irvine et al. 2010) where the energy generated by aerobic decomposition of compost (including green waste, industrial sludge, and liquid waste) in in-vessel composting tunnels over a 12-17 day period was investigated. While the study showed that 7,000-10,000 kJ kg⁻¹ of heat was generated for a 15 day composting period, use of the degraded compost material was limited to this time period only. In such processes, the feedstock of the process is a consumable item and energy harvested is related to the decomposition and consumption of the feedstock which in turn releases energy intrinsic to the feedstock.

Another problem in general with aerobic composting is that the temperature decreases over time due to changes in the microorganism community from predominantly thermophilic microorganisms (active composting stage) to predominantly mesophilic microorganisms (curing stage) as organic material is consumed. As a consequence, there is a relative short period of time where there are stable levels of biothermal energy generated during aerobic composting.

In addition, as the organic material is broken down during aerobic composting, carbon is lost as carbon dioxide.

It will be clearly understood that, if a prior art publication is referred to herein, this reference does not constitute an admission that the publication forms part of the common general knowledge in the art in Australia or in any other country.

### SUMMARY OF INVENTION

Embodiments of the present invention provide a method and system for the continuous generation and harvesting of energy from a source of biothermal energy, which may at least partially address one or more of the problems or deficiencies mentioned above or which may provide the public with a useful or commercial choice.

The term “biothermal energy” as used herein is broadly defined as the energy generated from the heat and/or gas by-products of biological reactions such as the decomposition or conversion of an organic material and the capture and storage of energy from an external energy source.

The term “catalyst” as used herein is broadly defined as a substance that produces or generates a reaction regardless of whether it undergoes a change itself.

The term “amendment” as used herein is broadly defined as a process or action that leads to a change in the condition of an organic material, including a physical change, a chemical change, a biological change, or any suitable combination thereof. In this instance, it will be understood that amending an organic material in effect amends the three-dimensional space including the surface of the organic material, the contiguous atmosphere about the organic material and the three-dimensional volume of the organic material below the surface of the organic material.

The term “dynamic equilibrium” as used herein is broadly defined as a state of a system which, in nutrient terms, consumption and regeneration of organic molecules is substantially in balance such that there is no net change in the quantum of organic material in the system. In such a state the total net energy input to the reaction and the total net energy output from the reaction are also relatively balanced such that the energy released by way of consumption of organic material is offset by energy captured in the re-composition of other organic structures and substances.

With the foregoing in view, the present invention in one form, resides broadly in a method for the continuous generation and harvesting of biothermal energy, the method comprising:
forming a heap comprising an amended organic material;
subjecting the amended organic material to a continuous fermentation process to produce a convection current and to stimulate the continuous capture of non-visible radiation, and
using a heat exchanger in contact with the heap to capture and/or store the biothermal energy generated by the continuous fermentation process within the heap.

Advantageously, the present invention enables the continuous generation and harvesting of biothermal energy generated by the continuous fermentation of an organic material. In addition, the continuous fermentation of the organic material generates heat within a consistent temperature range which may produce a reliable source of biothermal energy day and night. In addition, the continuous fermentation process includes both decomposition or conversion of organic structures and the re-composition of organic material, enabling the generation of the biothermal energy over a longer period of time compared to traditional composting methods. In addition, the continuous fermentation of the organic material continuously rebuilds a pool of high molecular weight organic molecules creating a dynamic equilibrium in nutrient terms, such that consumption and regeneration of the organic molecules are substantially in balance.

The present invention provides a method for continuous generation and harvesting of biothermal energy. In a preferred embodiment, the present invention provides a method for the continuous fermentation of an organic material which converts the organic material into a source of biothermal energy.

As indicated, the method for continuous generation and harvesting of biothermal energy comprises forming a heap comprising an amended organic material.

The amended organic material may be amended by any suitable process. Preferably, the organic material may be amended by applying one or more catalysts to the organic material.

In use, it is envisaged that the one or more catalysts may stimulate and promote the proliferation of desired microorganisms in and/or on the organic material, facilitating the continuous fermentation of the organic material and generating an amended organic material which includes the characteristics and elements commonly found in a humified soil.

Any suitable organic material may be used in the method. For instance, vegetable matter (including fruits, vegetables, pulses, grains, grasses, cane trash, straw, etc.) or animal matter (including an animal by-product material (such as an animal carcass, bone, fat, connective tissue, offal, blood, feathers, hair, fur, skin, horns, hooves, or the like), an animal manure or urine, a dairy waste material (such as whey, curds, or the like) may be used. The organic material may be fresh organic material, food scraps, waste material (including rotting food or other organic material such as green waste, paper, cardboard, wood chips, spoilt hay, silage, weeds, vineyard and orchard waste, timber mill waste), biosolids or the like, or a combination thereof.

In some embodiments, the organic material may include a mixture of materials that are rich in nitrogen (such as manure, urine, food waste, lawn clippings, etc.) and materials that are rich in carbon (such as straw, sawdust, green waste, paper, etc.). Preferably, the nitrogen-rich materials and carbon-rich materials may be mixed to form the organic material.

The organic material may be subjected to a size reduction process to produce a size-reduced organic material. Any suitable size reduction technique may be used.

For instance, the organic material may be crushed, ground, cut, milled, shredded, disintegrated, torn, or the like, or any combination thereof.

The organic material may be subjected to one or more size reduction processes. Any such size reduction processes may be completed in a single or multiple pass operation, which may include one, two, three, four, or any number of size reduction steps, to achieve a desired average particle size.

A person skilled in the art will appreciate that the length of time for which the organic material is subjected to the size reduction process may vary depending on a number of factors including the type of organic material, the volume of organic material, the type of size reduction technique being used, the preferred particle size of the size reduced organic material product and so on.

Although the size reduction process may be used for any organic material, it is envisaged that a size reduction process may be most beneficial where a proportion of the organic material is greater than 5 cm in size (for instance, branches, large bones, animal carcasses etc.).

Any suitable catalyst may be used. Generally, the catalysts may comprise a source of and/or a substrate produced by, and which stimulates the activity of, the one or more prokaryotic organisms. In this instance, it is envisaged that the catalyst may have the capacity to capture non-visible radiation and trigger phototrophic and/or phosphorolytic reactions such that the prokaryotic organisms may process the substrate and generate simple sugars.

For instance, the catalyst may provide a substrate which stimulates the activity of one or more species of Archaea, one or more species of bacteria, or any suitable combination thereof. The prokaryotic organism may be anaerobic, aerobic, autotrophic, heterotrophic, phototrophic, chemotrophic, photosynthetic, or any suitable combination thereof.

Generally, the catalyst provides a substrate which may stimulate the activity of low temperature fermentation microorganisms. The term “low temperature fermentation microorganisms” is a term of the art and includes microorganisms which may have fermentative activity at low temperatures. For example, the microorganism may have fermentative activity at temperatures of about 20 to 45° C. However, a person skilled in the art will appreciate that the temperature for fermentative activity may vary depending on a number of factors including the species of microorganism, and the type and condition of the substrate.

In some embodiments, the catalyst may stimulate the activity of at least one of an aerobic microorganism, an anaerobic microorganism, and a photosynthetic microorganism. Preferably, the catalyst may stimulate the activity of a heterotrophic photosynthetic bacteria and/or prokaryotic organism including either one of Archaea or bacteria.

In some embodiments, the catalyst may stimulate the activity of one or more prokaryotic organisms, such as heterotrophic photosynthetic bacteria, other phototrophic species, lactobacillus species, yeasts, actinomycetes species, Nocardia species, ray fungi, plankton, chemotrophic bacteria, autotrophic bacteria, or a suitable combination thereof.

In an embodiment of the invention, the catalyst may be mixed with one or more other substances before the catalyst may be applied to the organic material.

Any suitable type of substance may be used.

For instance, the substance may act as a processing aid for storage and delivery of the catalyst, may facilitate the application of the catalyst to the organic material, may facilitate the organic material taking up the catalyst, may maintain viability of a microorganism in the catalyst, increase the available pool of a nutrient in the organic material, may stimulate a targeted response in nutrient accumulation, or the like.

For instance, an additive such an emulsifier, a stabiliser, a wetting agent, a preservative, a surfactant, a mineral, a source of a nutrient, or the like, may be added. For instance, a source of calcium may be added to the catalyst to increase the available calcium in the organic material. For instance, a source of sugar (such as molasses), may be added to the catalyst to improve the fermentative capacity of the organic material.

In some embodiments, the catalyst may comprise a source of biologically available phosphorus.

The catalyst may comprise any suitable amount of biologically available phosphorus.

For example, the catalyst may comprise biologically available phosphorus of between about 0.1% w/v and 2% w/v, between about 0.5% w/v and 1 % w/v.

For example, the catalyst may comprise biologically available phosphorus of less than about 15% w/v, less than about 5% w/v, less than about 1 % w/v.

In some embodiments, a second catalyst comprising a source of biologically available phosphorus may be applied to the organic material. In this instance, it will be understood that there may be at least a first catalyst and a second catalyst.

In some embodiments, the first catalyst and the second catalyst may be the same type of catalyst, or may be different types of catalyst.

In some embodiments, the catalysts may perform the same type of function, or may have different functions.

Amendment of the organic material may be carried out in any suitable manner.

For instance, the one or more catalysts may be sprayed onto the organic material, may be drip irrigated, may be furrow irrigated, may be aerially applied, may be broadcasted or spread, or any suitable combination thereof. However, it will be understood that the method of applying the one or more catalysts to the organic material may vary depending on a number of factors, such as the composition and characteristics of the catalyst, the method of application, the type and amount of organic material to be treated, and the treatment regime.

The one or more catalysts may be applied to the organic material in any suitable manner. For instance, the catalysts may be stored and applied to the organic material together, may be stored in separate storage vessels and applied to the organic material together, or may be stored in separate storage vessels and applied to the organic material independently of one other. In an embodiment of the invention wherein the catalysts may be applied to the organic material independently of one another, it is envisaged that the catalysts may be applied sequentially to the organic material or may be applied to the organic material at the same time. However, it will be understood that the method of applying the catalysts to the organic material may vary depending on a number of factors, such as the composition and characteristics of the catalysts, the method of application and the type and amount of organic material to be treated.

The one or more catalysts may be added to the organic material before, during, or after the size reduction process.

In this instance, it will be understood that catalysts may be added to the organic material during preparation of the organic material for size reduction, prior to commencement of the size reduction process, during the size reduction process, after the size reduction process may be completed, or any suitable combination thereof.

Typically, however, the catalysts may be added to the size-reduced organic material during the size reduction process and before the size reduction process may be completed. However, a person skilled in the art will appreciate that the point of addition of the catalysts to the organic material during the size reduction process may vary depending on a number of factors, such as the type of size reduction process, the number of passes in the size reduction process, the type and composition of organic material and the type of the catalyst.

In some embodiments, the one or more catalysts may be added to the organic material before, during, or after formation of a heap of the organic material.

The amended organic material may have a relatively high initial moisture content.

Any suitable initial moisture content may be used. Generally, the initial moisture content may be sufficient to enable stable piling of the heap and prevent excessive leachate without stalling the fermentation process.

For example, the amended organic material may have an initial moisture content of between about 30% w/w and 90% w/w, between about 40% w/w and 80% w/w, between about 50% w/w and 70% w/w of the total mass of the amended organic material. Preferably, the amended organic material may have an initial moisture content of about 65 % w/w.

The desired initial moisture content of the amended organic material may be achieved by any suitable means.

For instance, a catalyst solution may be added to the organic material, water may be added either prior to or after the introduction of the catalyst to the organic material, organic materials having a low moisture content (such as paper-based materials, sawdust, woodchips, grain and dry hay) may be soaked using water sprays or immersed in water prior to being inoculated or any suitable combination thereof.

Advantageously, a relatively high moisture content may facilitate the spread of microorganisms in the catalyst through the heap. Thus, while the heap may be static, the circulation of water through the heap ensures that microorganisms constantly move throughout the heap, ensuring that the continuous fermentation process occurs throughout substantially the entire heap.

In some embodiments, the movement of the microorganisms throughout the heap may increase the kinetic energy of the water and/or particles in the heap creating a convection current.

The amended organic material may be formed into a heap using any suitable technique. For instance, the amended organic material may be manually formed into one or more heaps using tools such as shovels, and/or using one or more load-shifting machines, such as a backhoe, front end loader, tractor, or the like.

The heap may be formed to one or more specific sizes, volumes, or shapes. In a preferred embodiment of the invention, the heap may be formed so as to minimise the surface area of the heap while simultaneously maximizing the internal volume of the heap. Therefore, it is envisaged that a heap will be formed from as much amended organic material as possible.

For example, the heap may be formed as a windrow (i.e. an elongate heap having a greater length than width), a substantially frustoconical shape, a dome shape, or the like.

The heap may be of any suitable size. Generally, the height of the heap and the ratio of the heap height to width in cross-section may be sufficient to cause compression of the amended organic material under gravity alone and to increase the efficiency of convection within the heap. However, a person skilled in the art will appreciate that the exact dimensions of the windrow may vary depending on a number of factors, including the available space, the volume of organic material available and so on.

The height to width ratio of the heap in cross-section may be of any suitable dimension.

For example, the height to width ratio of the heap in cross section may be about 4:1, about 8:3, about 2:1. In some embodiments, the height to width ratio may be about 8:3.

In some embodiments, the minimum height of the heap may be at least about 2.5 metres with a preferred height to width ratio of about 8:3. In this instance, it is envisaged that the width of the heap may be at least about 95 cm.

In some embodiments, the heap may be constructed such that a concave shape is formed in the upper surface of the heap. In this instance, it is envisaged that the heap may be approximately an M-shape in cross-section.

The concave shape may assist in moisture reticulation throughout the heap. For instance, forming the heap in this manner may assist in establishing convection currents within the heap so that water that condenses on the cover over the heap will run into the concave recess in the upper surface of the heap and re-enter the amended organic material. In this way, water is substantially prevented from condensing at the edges of the heap and being lost as run off.

The concave shape may have any suitable depth. However, a person skilled in the art will appreciate that the depth may vary depending on a number of factors such as the size and shape of the heap, the type of organic material and the age of the heap.

For example, the height of the heap relative to the depth of the concave shape may be about 2:1, about 4:1, about 6:1, about 8:1, about 10:1. In some embodiments, the height of the heap relative to the depth of the concave shape may be about 10:1.

In some embodiments, the heap may have a minimum height of at least about 2.5 metres, a width of at least about 4 metres and a concave shape having a depth of at least about 40 cm.

In some embodiments, the heap may be provided with a cover. Generally, covering the heap may assist in maintaining the level of moisture within the heap during the continuous fermentation process, maintaining the stability of the temperature within the heap within the desired range despite external or ambient temperatures, reduce the impact of environmental issues (such as the creation of odours and dust), reduce infestation by vermin, and the like.

Any suitable cover may be used, such as one or more sheets of a waterproof material (such as tarpaulins, silage covers, or the like), a cap (such as a clay cap), or any suitable combination thereof.

By forming the heap in this manner, and providing a seal around the heap formed by the covers, the loss of moisture from the heap during the continuous fermentation process may be reduced or eliminated. Thus, the heap may be maintained at a desired moisture content throughout the continuous fermentation process without the need to add additional water to the heaps, or with the addition of only relatively small quantities of water.

In addition, the covering of the heaps may assist in maintaining the stability of the temperature within the heap within the desired range despite external or ambient temperatures. This assists in ensuring that the desired biological activity takes place generally throughout the heap without the need for mechanical mixing, and ensures that the rate of biological activity is maintained at a desired level throughout the heap.

The fermentation conditions of the heap comprising an organic material may promote the capture of specific wavelengths of electromagnetic radiation.

In some embodiments, the fermentation conditions of the heap stimulate the continuous capture of non-visible radiation.

In this instance, it will be understood that non-visible radiation comprises electromagnetic radiation having wavelengths that fall above and/or below visible light, that is, infrared light, violet or ultraviolet light, X-rays, radio waves, microwave, gamma rays and the like. Preferably, the fermentation conditions may promote activity of non-plant chlorophyll-based organisms and/or decreases activity of green and/or black sulphur bacteria. Advantageously, the method of the present invention results in fermentation of an organic material without requiring special environmental conditions.

As indicated, the method for continuous generation and harvesting of biothermal energy comprises subjecting the organic material to a continuous fermentation process to produce a convection current and to stimulate the continuous capture of non-visible radiation.

The amended organic material may be subjected to any suitable continuous fermentation process.

Preferably, the continuous fermentation process comprises aerobic, anaerobic and heterotrophic activity.

In use, it is envisaged that the continuous fermentation process may initiate bacterial photosynthesis (including both direct phototrophic and autotrophic activity) and secondary chemotrophic activity such that the net result is the capture of solar energy and storage of the captured energy as organic molecules.

In some embodiments, the continuous fermentation process may trigger nutrient accumulation, including, but not limited to, nitrogen and carbon sequestration. In use, it is envisaged that the stimulation of sequestration capacity may result in the continuous sequestration of nutrients from the amended organic material in the heap and/or from the surface of the heap, the contiguous atmosphere above the heap and the three-dimensional area of the heap below the surface of the heap.

The nutrient accumulation process subsequently results in the formation of a high molecular weight organic molecules, wherein the high molecular weight organic molecules may be an energy storage compound and a nutrient storage compound.

In some embodiments, the continuous fermentation process may be in a state of dynamic equilibrium. In this instance, it will understand that the continuous fermentation of the organic material continuously rebuilds a pool of high molecular weight organic molecules creating a dynamic equilibrium in nutrient terms, such that accumulation and turnover of the organic molecules may be substantially in balance.

Preferably, the high molecular weight organic molecules may constitute humus or a humic substance. Advantageously, the capture of energy and the generation of humus or humic substances results in a constant net production of heat energy.

Preferably, the continuous fermentation process occurs in a low oxygen environment. The term “low oxygen environment” is a term of the art and includes environments which have an oxygen content lower than the external gaseous environment and in which biological reactions are not dominated by, or dependent upon the presence of oxygen. For example, the amended organic material may have an oxygen content of below about 2 to 4 ppm. Without wishing to be bound by theory, it is believed that restricting the oxidation of the amended organic material assists in converting the amended organic material into humus or a humified soil without being decomposed into carbon dioxide.

In particular, biological hydrosynthesis (generation of additional moisture, increased water storage capacity of the material, and increased evapotranspiration within the heap), convection of moisture within the heap, compression of the amended organic material (by virtue of the height of the heap) to minimise air gaps and particle size reduction (either before piling up the heap and/or during the fermentation process) may assist in restricting the oxidation of the amended organic material. As a result, the temperature in the heap may be lower than expected where oxidation of the organic material predominates, but consistently about 5° C. to 20° C. above ambient temperature.

The continuous fermentation of the amended organic material may generate a source of energy. For instance, the continuous fermentation process may be exothermic and generate heat energy, may generate energy by breaking chemical bonds, may generate kinetic energy from the movement of microorganisms throughout the heap, may generate heat energy from the turnover of the humus by microorganisms in the heap, or any suitable combination thereof.

A convection current may be formed in any suitable manner. For instance, the convection current may be formed by movement of water and/or heat within the heap, by movement of microorganisms through the heap, by movement of nutrients and/or particles through the heap, or the like.

In use, it is envisaged that the convection current oscillates through the heap attracting and dispersing nutrients and facilitating the generation and dispersal of energy.

The amended organic material may be subjected to a continuous fermentation process for any suitable period of time. Preferably, the period of time will be sufficient to produce a stable convection current and thereby a source of biothermal energy.

However, a person skilled in the art will appreciate that the period of time required to produce a stable convection current may vary depending on a number of factors including the size of heap, the type of organic material used, the type of catalyst used, the quantity of catalyst used, the moisture content of the heap, environmental factors such as ambient temperature and so on.

For instance, the period of time required for the heap to produce stable convection current may be at least 24 hours, at least three days, at least five days, at least seven days, at least ten days.

In some embodiments, the heap produces a stable convection current at a temperature of about 40° C. to 65° C. and a moisture content of about 65% w/w.

In use, it is envisaged that, as the aerobic microbial activity within the one or more heaps begins to decline, and anaerobic microbial activity and fermentative processes become more dominant, the temperature of the one or more heaps may decrease and stabilise at temperatures of below about 65° C. While not critical, it is envisaged that the temperature may stabilise at between about 40° C. and 65° C. While dependent on a number of factors, it is envisaged that the temperature may stabilise at between about 40° C. and 65° C. between about three and ten days from the commencement of the fermentation process.

In some embodiments, the temperature of the heap may be substantially regulated by the moisture recirculating through the heaps. As moisture from the heap rises, condenses on the covers over the heap and is at least partially re-directed towards the centre of the heaps, the temperature may remain relatively constant through the heap. Thus, an effective mixing and generalised heating system may generate a convection current within the heap rather than being concentrated in one or more locations within the heap.

As indicated, the method for continuous generation and harvesting of biothermal energy comprises using a heat exchanger in contact with the heap to capture and/or store biothermal energy generated by the continuous fermentation process within the heap. Advantageously, the heap provides a reliable source of biothermal energy day and night at a consistent temperature range over a longer period of time compared to traditional composting methods increasing the cost effectiveness of harnessing biothermal energy.

Any suitable heat exchanger may be used.

Generally, the heat exchanger may be an indirect-contact heat exchanger wherein the heat transfer fluid and the moisture in the heap remain separate. For instance, the heat exchanger may be a direct transfer type exchanger or recuperator (such as tubular exchangers, plate-type exchangers and extended surface exchangers), a storage type exchanger (such as a regenerative heat exchanger or regenerator), or the like.

In some embodiments, the heat exchanger may be a low temperature and/or a waste heat exchanger. The term “low temperature heat exchanger” is a term of the art and refers to heat exchangers which operate at a low temperature differential relative to ambient temperature. For example, geothermal pool heating equipment requires only one to two degrees temperature differential relative to ambient temperature in order to extract the required geothermal energy.

For instance, the heat exchanger may be a plate-type exchanger, a regenerator, a heat pump, an economiser or finned tube heat exchanger, waste heat boiler, or the like.

The heat exchanger may contact the heap in any suitable manner. Generally, the contact between the heat exchanger and the heap may be sufficient so as to facilitate the efficient transfer of biothermal energy from the heap to the heat exchanger. However, a person skilled in the art would understand that the point of contact between the heat exchanger and the heap may vary depending on a number of factors, such as the type and size of heat exchanger and the size and configuration of the heap.

For instance, one or more components of the heat exchanger may be located in proximity to the heap, in contact with a surface of the heap, within the heap, underneath the heap, or any suitable combination thereof.

In some embodiments, one or more components of the heat exchanger may be located underneath the heap and/or within the heap.

The one or more components in contact with the heap may include heat transfer elements (such as a core or matrix containing the heat transfer surface) of the heat exchanger. The one or more components of the heat exchanger in contact with the heap may be in fluid communication with fluid distribution elements (such as headers, manifolds, tanks, inlet and outlet nozzles, etc.) located external to the heap.

For example, the heat exchanger may comprise a matrix or network of pipes located underneath and/or within the heap in fluid communication with a pumping and storage installation located external to the heap.

In some embodiments, the matrix of pipes may be provided with extended surface elements or fins, wherein heat from the heap may be transferred to the fin due to movement of water within the heap and conducted from the fin into the heat transfer fluid.

In some embodiments, the biothermal energy captured by and/or stored in the heat exchanger may be used as a source of power.

For instance, the heat exchanger may be associated with a heat driven generator, wherein the biothermal energy captured by the heat exchanger may be converted to mechanical work which may be used to run a generator to produce electricity.

For instance, the biothermal energy captured by the heat exchanger may be used to heat a fluid (such as a liquid or a gas), wherein the heated fluid is used to heat a structure, may be used to heat water for domestic use, or the like.

Any of the features described herein can be combined in any combination with any one or more of the other features described herein within the scope of the invention.

The reference to any prior art in this specification is not, and should not be taken as an acknowledgement or any form of suggestion that the prior art forms part of the common general knowledge.

### BRIEF DESCRIPTION OF DRAWINGS

Preferred features, embodiments and variations of the invention may be discerned from the following Detailed Description which provides sufficient information for those skilled in the art to perform the invention. The Detailed Description is not to be regarded as limiting the scope of the preceding Summary of Invention in any way. The Detailed Description will make reference to a number of drawings as follows:

FIG. 1 illustrates a perspective view of a system for the continuous generation and harvesting of energy from a source of biothermal energy according to an embodiment of the invention.

FIG. 2 illustrates a flowchart showing steps in a method for the continuous generation and harvesting of energy from a source of biothermal energy according to an embodiment of the invention.

### DETAILED DESCRIPTION

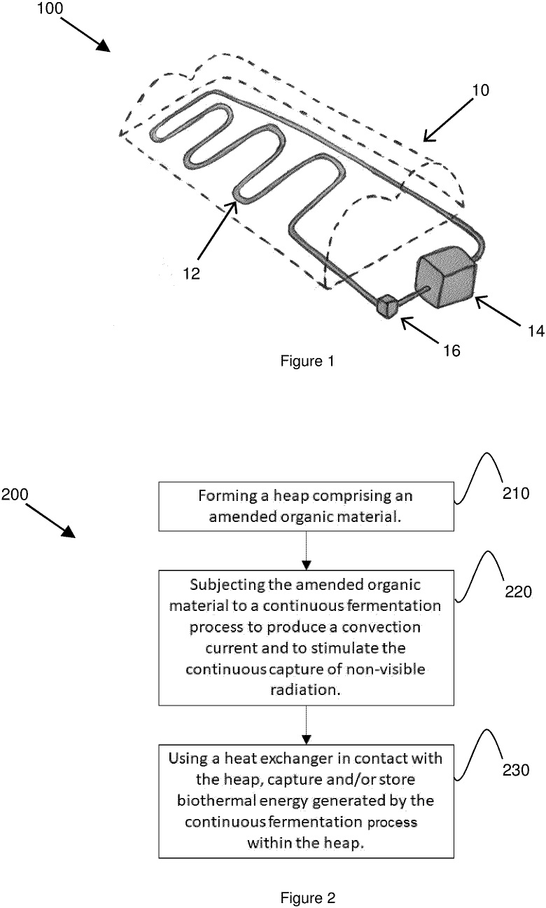

FIG. 1 illustrates a system for the continuous generation and harvesting of energy from a source of biothermal energy 100. Heap 10 may be formed from an amended organic material and may be approximately an M-shape in cross section. The amended organic material in the heap 10 may be subjected to a continuous fermentation process, the movement of microorganisms, moisture and heat through the heap may create a convection current and thereby a source of biothermal energy.

Heat exchanger includes a heat driven generator 14 and heat transfer elements 12 such as a network of pipes located underneath and/or within the heap 10. The network of pipes 12 may be in fluid communication with a pump 16 located external to the heap. In use, it is envisaged that biothermal energy generated by the heap 10 may be transferred to the network of pipes 12 due to movement of the water within the heap. Biothermal energy captured by the heat exchanger and used as a source of power.

A method for the continuous generation and harvesting of energy from a source of biothermal energy (200) is now described in detail with reference to FIG. 2.

At step 210, a heap comprising an amended organic material is formed.

Any suitable organic material may be used. Generally, however, the organic material may comprise vegetable matter or animal matter.

The organic material may be amended by applying one or more catalysts to the organic material.

The one or more catalysts may be applied to the organic material using any suitable technique.

The one or more catalysts may be applied to the organic material before, during or after a size reduction process and/or before, during, or after formation of a heap of the organic material.

In use, it is envisaged that the one or more catalysts may stimulate and promote the proliferation of desired microorganisms in and/or on the organic material, facilitating the continuous fermentation of the organic material and generating an amended organic material which includes the characteristics and elements commonly found in a humified soil.

Any suitable catalyst may be used. Generally, the catalysts may comprise a source of and/or a substrate produced by, and which stimulates the activity of, the one or more prokaryotic organisms.

In some embodiments, the catalyst may have the capacity to capture non-visible radiation and trigger phototrophic and/or phosphorolytic reactions such that the prokaryotic organisms may process the substrate and generate simple sugars.

Generally, the catalyst provides a substrate which may stimulate the activity of low temperature fermentation microorganisms.

In some embodiments, the catalyst may stimulate the activity of one or more prokaryotic organisms, such as heterotrophic photosynthetic bacteria, other phototrophic species, lactobacillus species, yeasts, actinomycetes species, Nocardia species, ray fungi, plankton, chemotrophic bacteria, autotrophic bacteria, or a suitable combination thereof.

In some embodiments, the catalyst may comprise a source of biologically available phosphorus.

In some embodiments, the amended organic material may have an initial moisture content of about 65 % w/w. Generally, the moisture content may be sufficient to facilitate the spread of microorganisms in the catalyst through the heap. Thus, while the heap may be static, the circulation of water through the heap ensures that microorganisms constantly move throughout the heap, ensuring that the continuous fermentation process occurs throughout substantially the entire heap.

The heap may be of any suitable size and cross-section. Generally, the height of the heap and the ratio of the heap height to width in cross-section may be sufficient to cause compression of the amended organic material under gravity alone and to increase the efficiency of convection within the heap.

In some embodiments, the heap may be constructed such that a concave shape is formed in the upper surface of the heap. Generally, the concave shape may assist in moisture reticulation throughout the heap.

In some embodiments, the heap may be provided with a cover.

The fermentation conditions of the heap comprising an organic material may promote the capture of specific wavelengths of electromagnetic radiation.

At step 220, the amended organic material is subjected to a continuous fermentation process to produce a convection current and to stimulate the continuous capture of non-visible radiation.

Preferably, the continuous fermentation process comprises aerobic, anaerobic and heterotrophic activity. In use, it is envisaged that the continuous fermentation process may initiate bacterial photosynthesis (including both direct phototrophic and autotrophic activity) and secondary chemotrophic activity such that the net result is the capture of solar energy and storage of the captured energy as organic molecules.

In some embodiments, the continuous fermentation process may be in a state of dynamic equilibrium.

The continuous fermentation of the amended organic material may generate a source of energy. For instance, the continuous fermentation process may be exothermic and generate heat energy, may generate energy by breaking chemical bonds, may generate kinetic energy from the movement of microorganisms throughout the heap, may generate heat energy from the turnover of the humus by microorganisms in the heap, or any suitable combination thereof.

A convection current may be formed in any suitable manner. For instance, the convection current may be formed by movement of water and/or heat within the heap, by movement of microorganisms through the heap, by movement of nutrients and/or particles through the heap, or the like.

In use, it is envisaged that the convection current oscillates through the heap attracting and dispersing nutrients and facilitating the generation and dispersal of energy.

At step 230, a heat exchanger in contact with the heap is used to capture and/or store biothermal energy generated by the continuous fermentation process within the heap.

In some embodiments, the heat exchanger may be a low temperature and/or a waste heat exchanger.

The heat exchanger may contact the heap in any suitable manner. Generally, the contact between the heat exchanger and the heap may be sufficient so as to facilitate the efficient transfer of biothermal energy from the heap to the heat exchanger.

In some embodiments, one or more components of the heat exchanger may be located underneath the heap and/or within the heap. For example, the heat exchanger may comprise a matrix or network of pipes located underneath and/or within the heap in fluid communication with a pumping and storage installation located external to the heap.

In the present specification and claims (if any), the word ‘comprising’ and its derivatives including ‘comprises’ and ‘comprise’ include each of the stated integers but does not exclude the inclusion of one or more further integers.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. A method for continuous generation and harvesting of biothermal energy comprising:
forming a heap comprising an amended organic material;
subjecting the amended organic material to a continuous fermentation process to produce a convection current and to stimulate continuous capture of non-visible radiation; and
using a heat exchanger in contact with the heap, at least one of capturing or storing biothermal energy generated by the continuous fermentation process within the heap.

2. A method for continuous generation and harvesting of biothermal energy according to claim 1, wherein an initial moisture content of the heap is about 65% w/w.

3. A method for continuous generation and harvesting of biothermal energy according to claim 1, wherein the amended organic material is formed by applying one or more catalysts to an organic material.

4. A method for continuous generation and harvesting of biothermal energy according to claim 3, wherein the one or more catalysts stimulate activity of one or more low temperature fermentation microorganisms.

5. A method for continuous generation and harvesting of biothermal energy according to claim 3, wherein the one or more catalysts stimulate autotrophic activity.

6. A method for continuous generation and harvesting of biothermal energy according to claim 1, further comprising:
applying a cover to the heap comprising the amended organic material, wherein the cover assists in maintaining the heap at a desired moisture content and temperature level.

7. A method for continuous generation and harvesting of biothermal energy according to claim 1, wherein the continuous fermentation process occurs in a low oxygen environment.

8. A method for continuous generation and harvesting of biothermal energy according to claim 1, wherein the heat exchanger is a low temperature heat exchanger.

9. A method for continuous generation and harvesting of biothermal energy according to claim 1, wherein the heat exchanger comprises a matrix of pipes located at least one of underneath or within the heap.
